(19) United States
(12) Patent Application Publication
(10) Pub. No.: US 2019/0307912 A1
(43) Pub. Date: Oct. 10, 2019

(54) FRAGRANCE DELIVERY DEVICE, SYSTEM, AND METHOD

(71) Applicant: TAKASAGO INTERNATIONAL CORPORATION, Tokyo (JP)

(72) Inventors: Thomas F. Santini, Doylestown, PA (US); Anthony R. Budraitis, Lambertville, NJ (US)

(73) Assignee: TAKASAGO INTERNATIONAL CORPORATION, Tokyo (JP)

(21) Appl. No.: 15/769,100

(22) PCT Filed: Oct. 20, 2016
(86) PCT No.: PCT/US2016/057910

§ 371 Date: Apr. 18, 2018

Related U.S. Application Data
(60) Provisional application No. 62/243,965, filed on Oct. 20, 2015.

(51) Int. Cl. A61L 9/03; A01M 1/20; A61L 9/012

(52) U.S. Cl. CPC A61L 9/03; A01M 1/2072; A01M 1/2077; A61L 2209/133; A01M 1/2088; A61L 9/012; A61L 2209/131; A01M 1/2083

(57) ABSTRACT

A fragrance delivery device, system, and method of use and manufacturing are disclosed. The device has a vapor releasing membrane including a plurality of micropores, wherein each respective micropore has a consistent diameter dimension in both a delivery condition and in a use-condition of the fragrance delivery device. The device further includes a flexible impermeable barrier coupled to the vapor releasing membrane along a perimeter thereof, wherein the vapor releasing membrane and impermeable barrier define a cavity there between having a predetermined volume. A substance is disposed in the cavity up to the predetermined volume, the substance comprising a volatile fragrance disposed in a matrix, wherein the substance has a first viscosity in the delivery condition and a second viscosity in the use-condition, the volatile fragrance being releasable from the matrix when exposed to a predetermined temperature and emittable through the plurality of micropores.

### CROSS-REFERENCE TO RELATED APPLICATION

This application is a U.S. National Stage Patent Application under 35 U.S.C. § 371 of International Application No. PCT/US2016/057910, filed on Oct. 20, 2016, which claims priority to U.S. Provisional Application No. 62/243,965, filed on Oct. 20, 2015, the contents of each of which are incorporated herein by reference in their entirety.

### BACKGROUND OF THE DISCLOSED SUBJECT MATTER

### Field of the Disclosed Subject Matter

The present disclosure relates to a fragrance delivery device, system, method of use and method of manufacture. The device according to the disclosed subject matter can be used as an alternative to wax melt devices or plug-in cartridges for purposes of example.

### Description of the Related Art

Wax melt devices, also commonly referred to as “wax or candle tart”, “wax potpourri melts”, “candle melts”, and “scent chips” are scented waxes in various shapes, without a wick, intended to be melted and contained in a potpourri burner, dish, or other similar warming unit. The fragranced wax melt, when placed on the warming unit and exposed to heat, melts into a dish, and with the benefit of the heat, the fragrance is released from the molten pool of hot wax contained within the dish into the surrounding air. These devices have become a popular alternative to the use of candles, in part due to safety concerns surrounding candle use. Unlike candles, warmer units generally do not require an open flame. Thus, certain concerns surrounding the safety of children and pets or forgetting to extinguish the candle are diminished. Further, candle sooting is also not a concern with such devices.

However, certain shortcomings limit an even more widespread use of wax melts by users. More particularly, once the wax melt is warmed, it becomes an exposed pool of hot molten wax, similar to that of a candle, and requires a level, horizontal orientation such that the pool of wax remains in the confines of the warming dish. Thus, many of the concerns related to the potential for spillage and safety when used in homes with children or pets that apply to candles remain true for wax melts. Post use, there is also the need to remove the wax, either in its hot molten form or after it has cooled and rehardened to prevent an unsightly wax pile remaining in the warming unit.

Further, like wax candles, fragrance loadings of wax melts are limited by the need to keep the wax melt in rigid form to retain its shape and allow for handling by the user. This may result in a less than optimal fragrance experience for the user, especially over the course of time the wax melt is utilized. Wax melts and other of such devices are further limited in that the melts are not easily interchangeable. As such, melts often must be used in their entirety prior to the use of another melt of different fragrance, or the melt has to be “scooped” out of a warming device resulting in potential waste of product. Such wax melt devices are further unable to be combined with another wax melt device to enable a user to create a personalized fragrance experience.

Therefore, there remains a need to provide a fragrance delivery device which provides the benefits of candles or wax melts, while improving upon at least such deficiencies. More particularly, a fragrance delivery device which may be more safely used near children and pets, provides for more efficient clean-up after use, and can provide an optimal fragrance experience over the course of its use is desired. The present disclosure addresses these and other needs in further detail below.

### SUMMARY OF THE DISCLOSED SUBJECT MATTER

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

The present disclosure relates to a fragrance delivery device, system, method of use, and method of manufacture, which alleviates many of the negatives associated with the use of wax melters and wax melts, and eliminates the open molten pool of hot liquid wax that is basic to their functionality. According to the disclosed subject matter, a self-contained fragrance delivery device is provided. The fragrance delivery device also provides for a vapor releasing membrane, which allows for a rate controlled dispersal of the fragrance into the surrounding environment and provides a longer lasting experience for the user in comparison with conventional devices. The disclosed subject matter allows for the manufacture of a reduced sized cartridge device, such that two devices of different fragrance types can be used at the same time in a single warming dish, to offer the user creative control and a personalized fragrance experience. Furthermore, the self-contained fragrance delivery device also permits the flexibility for the device to be used with a warming unit having a non-horizontal warming surface as the delivery devices according to the disclosed embodiments do not have spillage concerns as with conventional devices. As such, the device can be oriented in any configuration and not limited to the horizontal orientation required by the current wax melters. Further, the fragrance delivery device can be used with a warming unit plugged into an electrical outlet on a vertical wall. As such, more compact and efficient warming units can be utilized with the currently disclosed device as compared to the warming units dedicated for use with currently available wax melts.

To achieve these and other advantages and in accordance with the purpose of the disclosed subject matter, as embodied and broadly described, the disclosed subject matter includes a fragrance delivery device, comprising: a vapor releasing membrane including a plurality of micropores, wherein each respective micropore has a consistent diameter dimension in both a delivery condition and in a use-condition of the fragrance delivery device; a flexible impermeable barrier coupled to the vapor releasing membrane along a perimeter thereof, wherein the vapor releasing membrane and impermeable barrier define a cavity there between having a predetermined volume; and a substance disposed in the cavity up to the predetermined volume, the substance comprising a volatile fragrance disposed in a matrix, wherein the substance has a first viscosity in the delivery condition and a second viscosity in the use-condition, the volatile fragrance being releasable from the matrix when exposed to a predetermined temperature and emittable through the plurality of micropores in the use-condition.

In accordance with another aspect of the disclosed subject matter, a fragrance delivery system is disclosed comprising a delivery device as disclosed herein, and a warming unit to emanate energy to the fragrance delivery device at a predetermined temperature, wherein the volatile fragrance is releasable from the matrix when exposed to the predetermined temperature and emittable through the plurality of micropores in the use-condition. In accordance with another aspect of the disclosed subject matter, a method of using a fragrance delivery system is further disclosed.

In accordance with another aspect of the disclosed subject matter, a method of manufacturing a fragrance delivery device is disclosed, comprising: providing vapor releasing membrane including a plurality of micropores, wherein each respective micropore has a consistent diameter dimension in both a delivery condition and in a use-condition of the fragrance delivery device; coupling a flexible impermeable barrier to the vapor releasing membrane, wherein the vapor releasing membrane and impermeable barrier define a cavity there between having a predetermined volume; and disposing a substance in the cavity up to the predetermined volume, the substance comprising a volatile fragrance disposed in a matrix; and sealing the vapor releasing membrane with the flexible impermeable barrier at a perimeter thereof, wherein the substance has a first viscosity in the delivery condition and a second viscosity in the use-condition, the volatile fragrance being releasable from the matrix when exposed to the predetermined temperature and emittable through the plurality of micropores in the use-condition.

### BRIEF DESCRIPTION OF THE DRAWINGS

The subject matter of the application will be more readily understood from the following detailed description when read in conjunction with the accompanying drawings, in which:

FIG. 1A depicts an exterior top perspective view of a filled, sealed fragrance delivery device with a scent name “Lavender Fields” printed thereon, in accordance with an embodiment of the disclosed subject matter.

FIG. 1B depicts an exterior top perspective view of a filled, sealed fragrance delivery device with a random pattern of micropores, in accordance with an embodiment of the disclosed subject matter.

FIG. 2A depicts a cross-sectional perspective view of a fragrance delivery device in accordance with an embodiment of the disclosed subject matter.

FIG. 2B depicts an exploded view of the components of the device of FIG. 2A, according to the disclosed subject matter.

FIG. 2C depicts an exploded view of the components of a fragrance delivery device of another embodiment, according to the disclosed subject matter.

FIG. 3A depicts a cross-sectional perspective view of a fragrance delivery device with the un-shown side being a mirror image thereof, in accordance with another embodiment of the disclosed subject matter.

FIG. 3B depicts a cross-sectional perspective view of a fragrance delivery device with the un-shown side being a mirror image thereof, in accordance with another embodiment of the disclosed subject matter.

FIG. 4A depicts an exterior perspective view of a fragrance delivery system with a fragrance delivery device and a warming unit, in accordance with an embodiment of the disclosed subject matter.

FIG. 4B depicts a cross-sectional view of the system of FIG. 4A, according to the disclosed subject matter.

FIG. 5A shows a cross-sectional perspective view of two fragrance delivery devices in accordance with another embodiment of the disclosed subject matter.

FIG. 5B depicts a cross-sectional view of a system having a warming unit and the devices of FIG. 5A, according to the disclosed subject matter.

FIG. 6A shows a perspective view of two fragrance delivery devices with a warming unit, in accordance with another embodiment of the disclosed subject matter.

FIG. 6B shows another warming unit according to the disclosed subject matter.

FIG. 7A depicts a perspective view of an embodiment of the disclosed subject matter having a system with a device partially inserted into a wall mounted heater unit equipped with a fan, according to the disclosed subject matter.

FIG. 7B and FIG. 7C depicts a front and rear perspective views of the device and unit shown in FIG. 7A, according to the disclosed subject matter.

FIG. 7D depicts a front perspective view of an embodiment of the disclosed subject matter having a system with a device partially inserted into a wall mounted heater unit, according to the disclosed subject matter.

FIG. 7E depicts a rear perspective of the warming unit shown in FIG. 7D, according to the disclosed subject matter.

FIG. 8 depicts a cross-sectional perspective view of the device of FIG. 2A without a substance therein, according to the disclosed subject matter.

FIG. 9A depicts a perspective view of a fragrance delivery device, according to the disclosed subject matter.

FIG. 9B depicts a top plan view of a fragrance delivery device, according another embodiment of the disclosed subject matter.

### DETAILED DESCRIPTION

### Definitions

The terms used in this specification generally have their ordinary meanings in the art, within the context of this disclosure and in the specific context where each term is used. Certain terms are discussed below, or elsewhere in the specification, to provide additional guidance to a person of ordinary skill in the art describing the compositions and methods of the disclosure and how to make and use them.

As used herein, the use of the word “a” or “an” when used in conjunction with the term “comprising” in the claims and/or the specification may mean “one,” but it is also consistent with the meaning of “one or more,” “at least one,” a plurality, and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “microporous” means average pore sizes not exceeding about 1μm.

### Fragrance Delivery Device

Reference will now be made in detail to various embodiments of the disclosed subject matter, non-limiting examples of which are illustrated in the accompanying drawings. The device presented generally is intended for releasing a volatile active material, such as a fragrance, into the surrounding environment.

According to an embodiment of the disclosed subject matter, a fragrance delivery device is provided, comprising: a vapor releasing membrane including a plurality of micropores, wherein each respective micropore has a consistent diameter dimension in both a delivery condition and in a use-condition of the fragrance delivery device; a flexible impermeable barrier coupled to the vapor releasing membrane along a perimeter thereof, wherein the vapor releasing membrane and impermeable barrier define a cavity there between having a predetermined volume; and a substance disposed in the cavity up to the predetermined volume, the substance comprising a volatile fragrance disposed in a matrix, wherein the substance has a first viscosity in the delivery condition and a second viscosity in the use-condition, the volatile fragrance being releasable from the matrix when exposed to a predetermined temperature and emittable through the plurality of micropores in the use-condition.

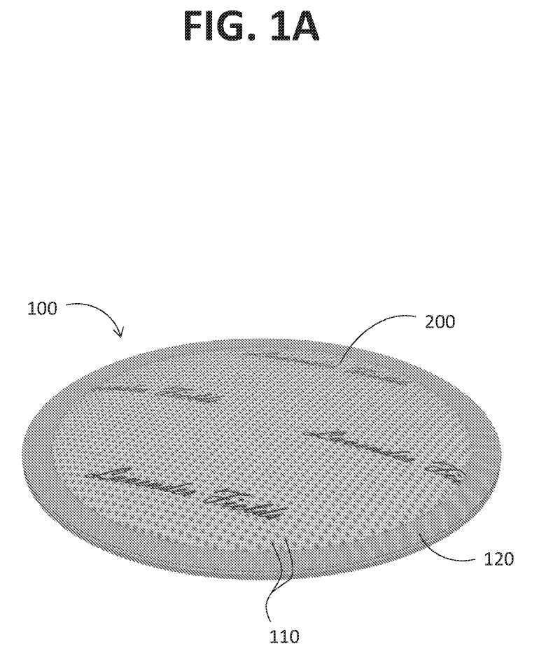

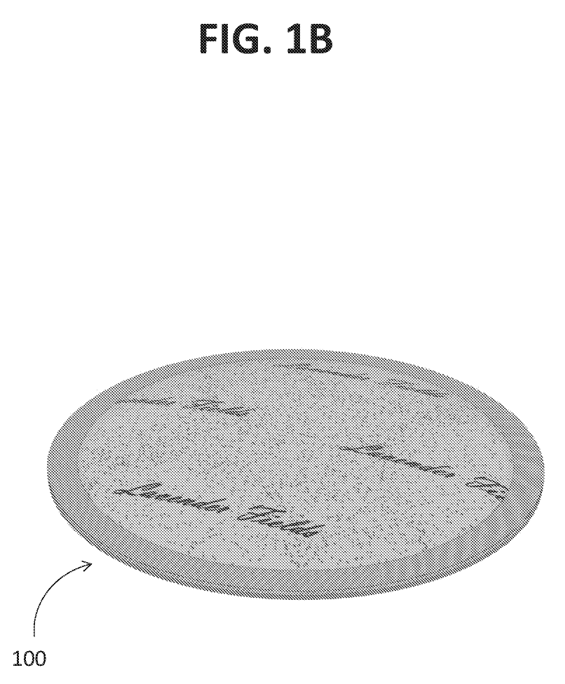

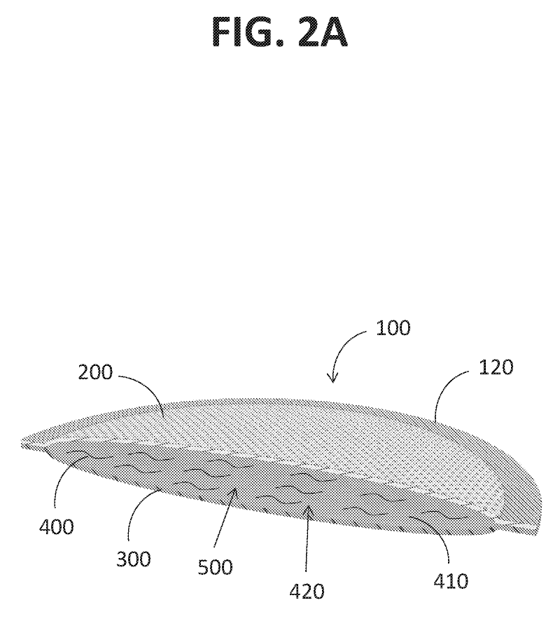

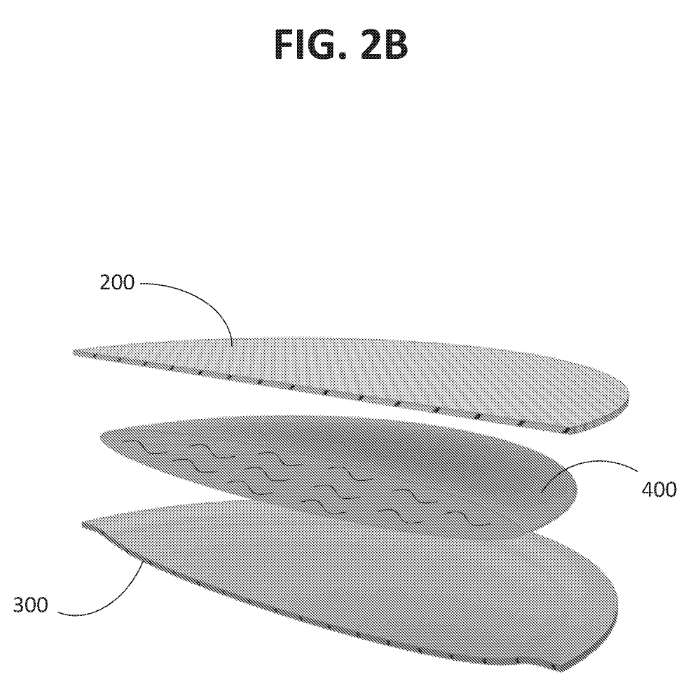

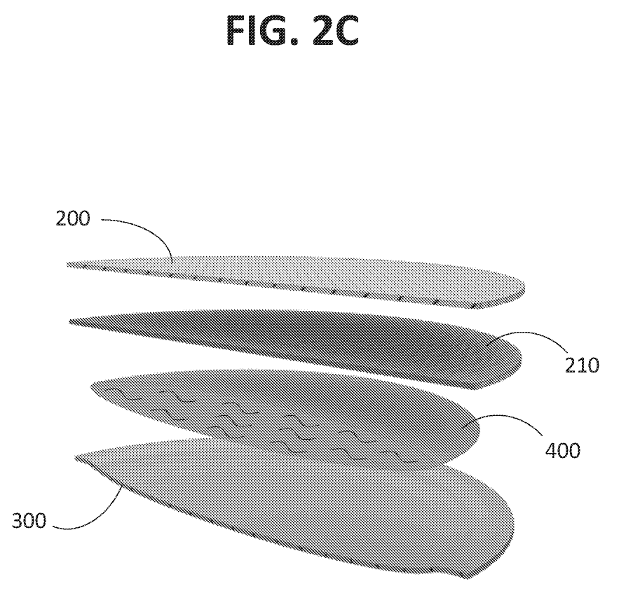

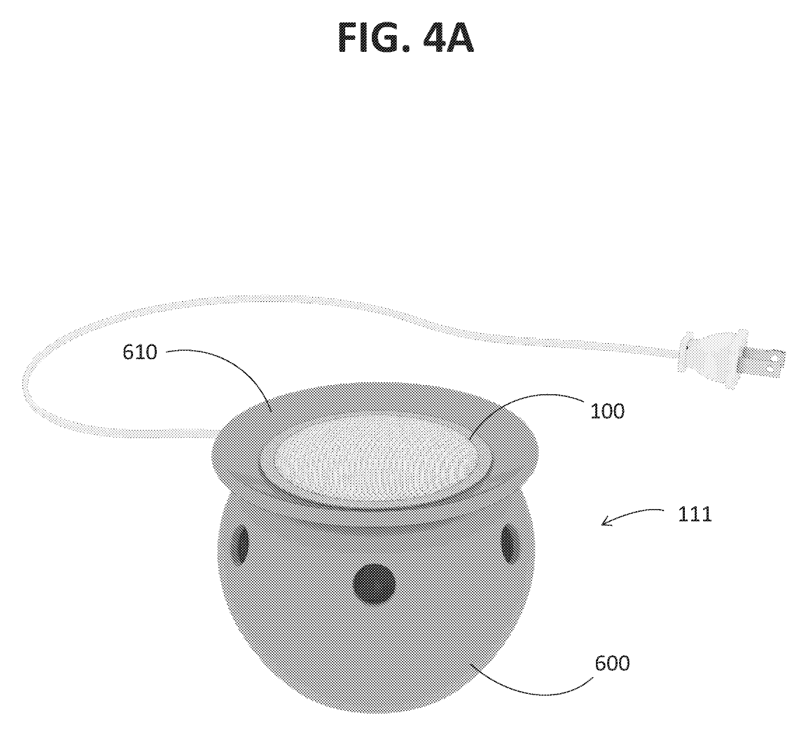

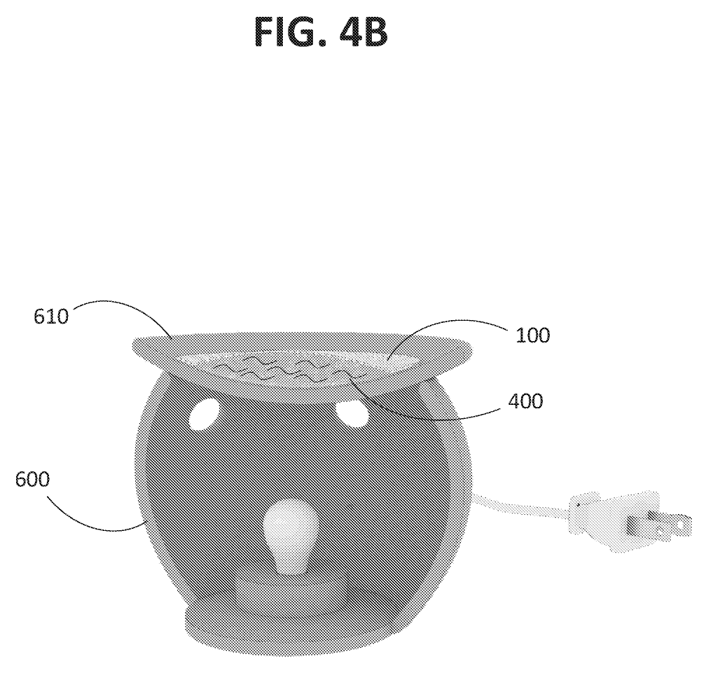

For the purpose of explanation and illustration, and not limitation, an embodiment of the fragrance delivery device 100 having a vapor releasing membrane 200, a flexible impermeable barrier 300, and a substance 400 therein is shown in FIGS. 1A-1B and 2A-2C. FIG. 1A and FIG. 1B depict fragrance delivery devices in top perspective views. FIG. 2A depicts cross-sectional view of a fragrance delivery device and FIG. 2B is an exploded view of the fragrance delivery device of FIG. 2A. FIG. 2C depicts an alternative delivery device in an exploded view as further discussed herein. The fragrance delivery device can be used with a warming unit to emit a volatile fragrance to a surrounding environment, as shown in FIG. 4A and FIG. 4B and further discussed herein.

The fragrance delivery device 100 includes a delivery condition when the device is not being used, and a use-condition when the device is actively being used with a warming unit. In the delivery condition, the vapor releasing membrane 200 of the fragrance delivery device 100 inhibits a volatile fragrance from emitting there through. In some embodiments, a negligible amount of volatile fragrance can be emitted through the vapor releasing membrane in the delivery condition. The delivery condition includes temperatures below the temperature of the warming unit. In the use condition, the volatile fragrance of the fragrance delivery device is freely emittable through the vapor releasing membrane 200 when exposed to a predetermined temperature, as further discussed herein.

The device 100 shown in FIG. 1A has a generally round shape and convex structure, which complements the shape of the top of the warming unit 600 as shown in FIG. 4A. However in accordance with the disclosed subject matter, the device may be made in a variety of geometric shapes (i.e. square, rectangular, hexagonal, octagonal, triangular, etc.). For example, the shape of the fragrance delivery device can be sized to better fit within the particular warming unit being utilized for optimum surface area exposure of the fragrance delivery device to a heated surface thereof. Alternatively, the fragrance delivery device can be shaped for design reasons, including but not limited to providing the user with a more aesthetically pleasing shape, without departing from the scope of the disclosed subject matter. The fragrance delivery device can furthermore have a variety of shapes when viewed in a side view, including but not limited to elliptical and substantially planar.

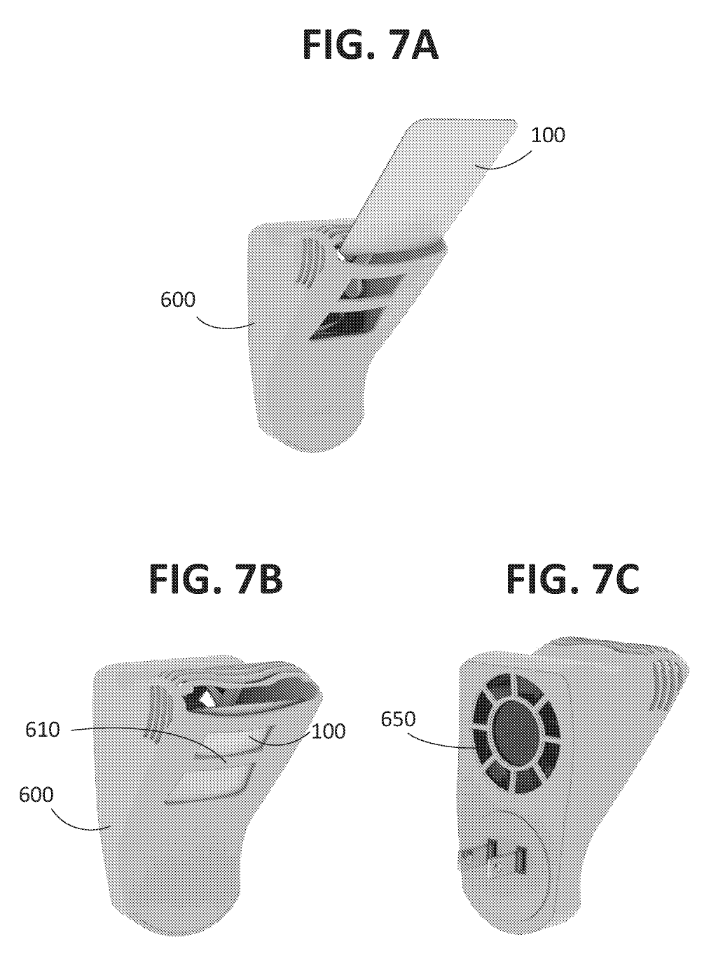

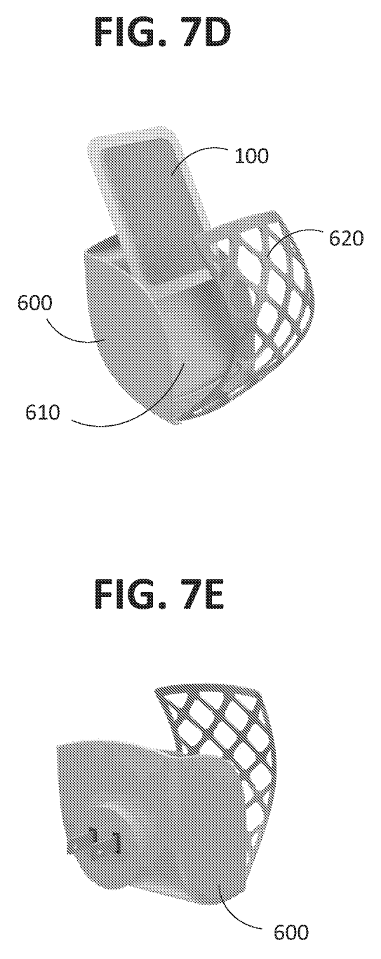

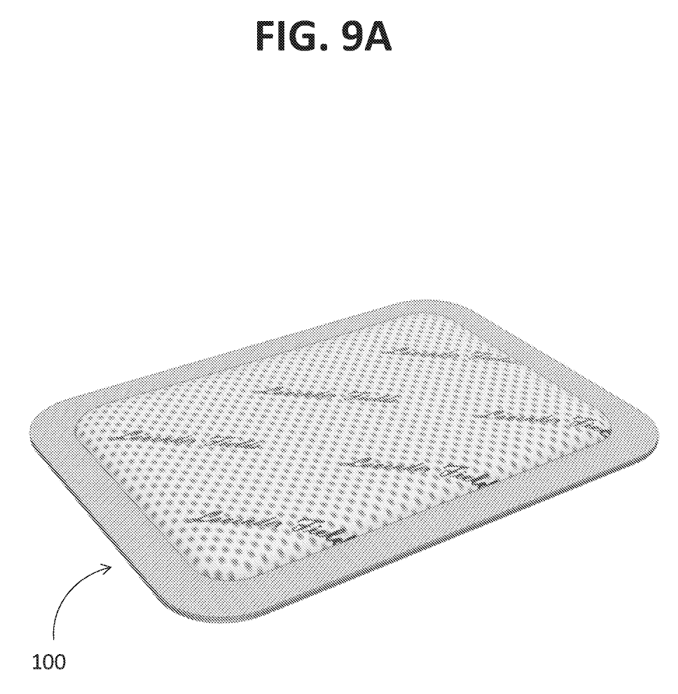

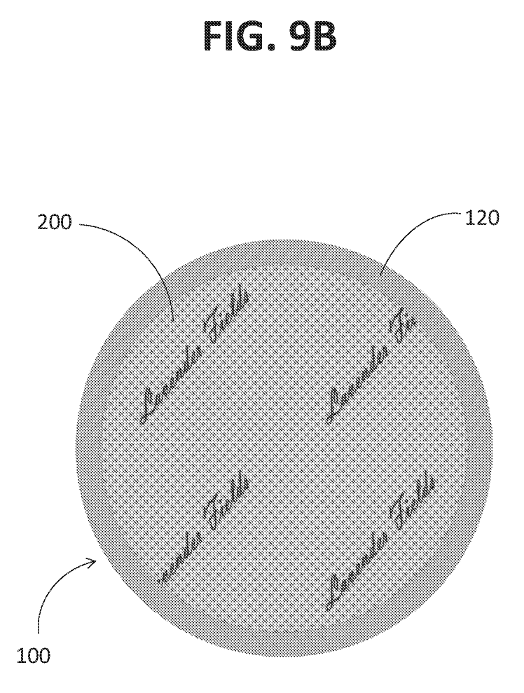

In the embodiment of FIGS. 7A-7E and 9A, the fragrance delivery device is rectangular to fit with an alternative wall-mounted warming unit having an angled placement surface as shown. The unit can be directly plugged into a wall outlet. The lack of any restrictions on the orientation of the fragrance delivery device allows for the manufacture and sale of warming units, such as plug-in formats as shown in FIGS. 7A-7E, that heretofore would not have been possible using the wax melt technology currently offered. In this embodiment, the warming unit additionally utilizes a small fan 650 built into the warmer unit 600 to assist in the dispersion of the volatile fragrance into the surrounding air and external environment. FIGS. 7B and 7C show the fragrance delivery device 100 fully inserted into the wall mounted warmer unit 600, incorporating a fan 650. Such embodiment illustrates a compact nature of the warming unit. The ability of the device to contain the volatile fragrance of the substance sealed within the device, as the volatile fragrance transitions from the delivery condition at ambient room temperature to a vapor state in the use-condition, affords an alternative format and flexibility to orient the device as desired (i.e. non-horizontal, horizontal, etc.) in comparison with wax melters.

FIG. 7D depicts a front perspective view of an embodiment of the disclosed subject matter having a system with a rectangular device partially inserted into a wall mounted heater unit, according to the disclosed subject matter. As shown, the warming unit 600 can additionally include a grille 620 to prevent accidental contact with the warming dish. The grille 620 can have a hinged connection at a first end and can lock into place at a second end. Such configuration can allow the grille 620 to act as a barrier to others, such as children and pets. FIG. 7E depicts a rear perspective of the warming unit shown in FIG. 7D, according to the disclosed subject matter. In the embodiment of FIG. 7D and 7E, a fan is not provided. The fragrance delivery device of FIG. 9A shows another embodiment showing the device in a rectangular pouch-like configuration that can be used with both horizontal or non-horizontal warming unit bases.

Turning back to FIG. 1A, the fragrance delivery device includes a vapor releasing membrane 200 positioned at a top portion thereof. As shown at least in FIG. 1A, the vapor releasing membrane 200 includes a plurality of micropores 110 for emission of the volatile fragrance there through in the use-condition. It is noted that the micropores 110 are not drawn to scale and shown throughout in the figures for purposes of illustration. As previously noted, each of the plurality of micropores does not exceed approximately 1 μm. In one embodiment, each respective micropore has a consistent diameter dimension in both the delivery condition and in the use-condition of the fragrance delivery device such that the diameter dimension remains unchanged between the delivery condition and the use-condition. As such, the vapor releasing membrane 200 is stable and unaffected by direct or indirect exposure to the predetermined temperature of a warming unit. In other embodiments, the vapor releasing membrane can be minimally altered at elevated temperatures, such as exposed to temperatures in excess of approximately 190° F., such that the membrane shrinks approximately 5% in a cross direction thereof and nominally shrinks in a machine direction thereof. The diameter dimension of each respective micropore can include any suitable dimension not to exceed 1 μm, such as for example approximately 0.05 μm and 0.06 μm. The vapor releasing membrane can further include any suitable thickness dimension that still permits the membrane to function as disclosed herein. In one embodiment, the membrane has a thickness of approximately 1 mil.

The plurality of micropores can be disposed in any suitable-pattern throughout the vapor releasing membrane and a pore size selected depending on a desired rate of release of the volatile fragrance there through in the use-condition. In the embodiment of FIG. 1A, the pattern of the plurality of micropores is uniform, which allows for uniform emissions of the volatile fragrance from the fragrance delivery device. In the embodiment of FIG. 1B, the pattern of the plurality of micropores is random. The vapor releasing membrane includes at least two functions—to inhibit the volatile fragrance from emitting through the plurality of micropores in the delivery condition and to permit the volatile fragrance to be emittable-through the plurality of micropores in the use-condition, as further discussed herein. As demonstrated by FIG. 2C, a non-woven substrate 210 can support the vapor releasing membrane to provide further structure thereto and be coupled therewith. The non-woven substrate can further include any suitable thickness dimension that still permits the device to function as disclosed herein. In one embodiment, the non-woven substrate thickness dimension ranges between and including approximately 2 mils to 4 mils and can have a basis weight of approximately 20 grams per square meter.

As depicted in FIG. 1A and FIG. 2A, the flexible impermeable barrier 300 is coupled to the vapor releasing membrane 200 along a perimeter thereof. As such, a perimeter seal flange 120 is formed by the joining of the vapor releasing microporous membrane 200 with the flexible impermeable barrier along a perimeter of the device shape. The flange 120 can be utilized as a gripping surface to place the fragrance delivery device on a warming unit and to remove the fragrance delivery device from the warming unit. Although the entire fragrance delivery device when in the use-condition is still able to be handled by a user, the flange provides an additional surface that is available for handling by the user. As depicted in the embodiment of FIG. 2A, the flange 120 can be raised above the base of the flexible barrier such that the flange 120 does not interface with a warming surface there below. As such, in those embodiments, the flange 120 has a cooler surface than the base of the flexible barrier that interfaces with the warming surface. In other embodiments, the delivery device is substantially planar such that the flange 120 can interface with the warming unit. Thus, in such embodiments, the flange can include the same temperature as the flexible barrier in the use condition.

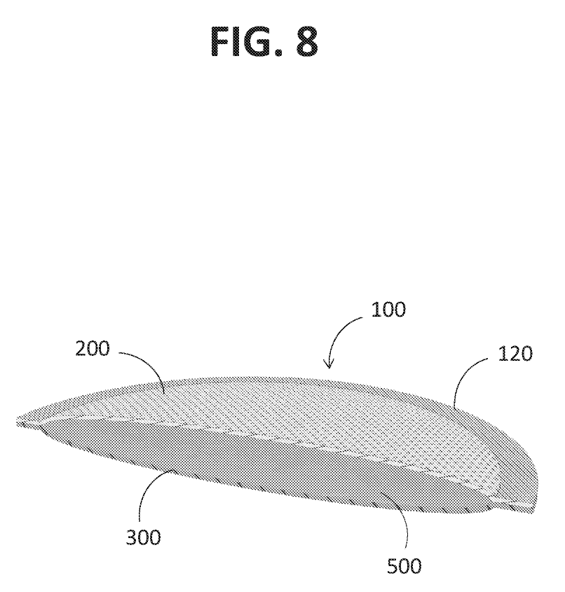

In FIG. 2A and FIG. 8, the cross-sectional views of the fragrance delivery device 100 depicts the membrane 200 and flexible impermeable barrier 300 coupled together to define a cavity 500 there between. FIG. 8 depicts a cross-sectional perspective view of the device of FIG. 2A showing the cavity 500 without the substance 400 therein, according to the disclosed subject matter. The cavity 500 has a predetermined volume that houses the substance 400 therein. The predetermined volume of the cavity can range depending on the desired fragrance life of the delivery device, such as for example from approximately 4.5 cc to approximately 28 cc, and capable of containing approximately 4 grams to approximately 25 grams of a substance, such as a gel substance. The substance 400 can be disposed in the cavity up to the predetermined volume. In the embodiment of FIG. 2A, the substance comprises the predetermined volume in the delivery condition, as such no head space is required between the lower flexible impermeable barrier 300 and the vapor releasing membrane 200. The lack of head space within the cavity facilitates optimum conditions for the substance to be uniformly heated and for the volatile fragrance within the matrix to transition into a vapor state and to emit through the plurality of micropores in the use condition.

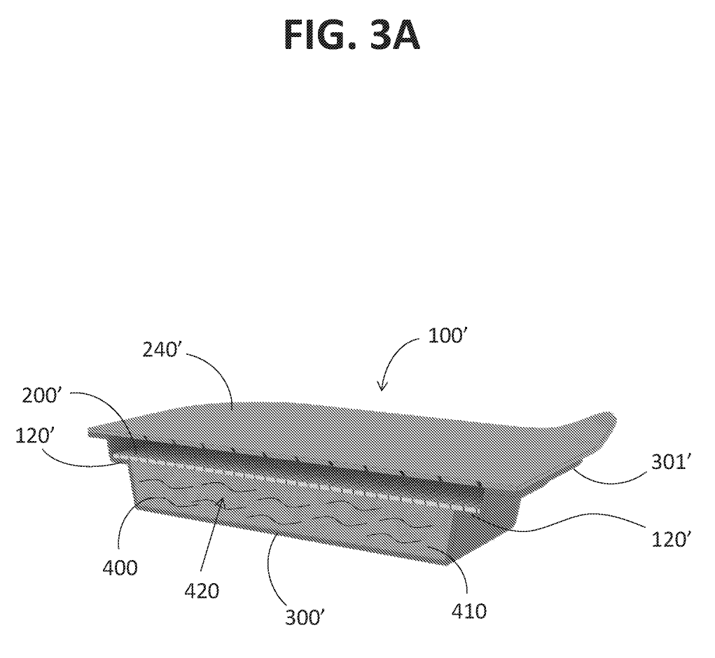

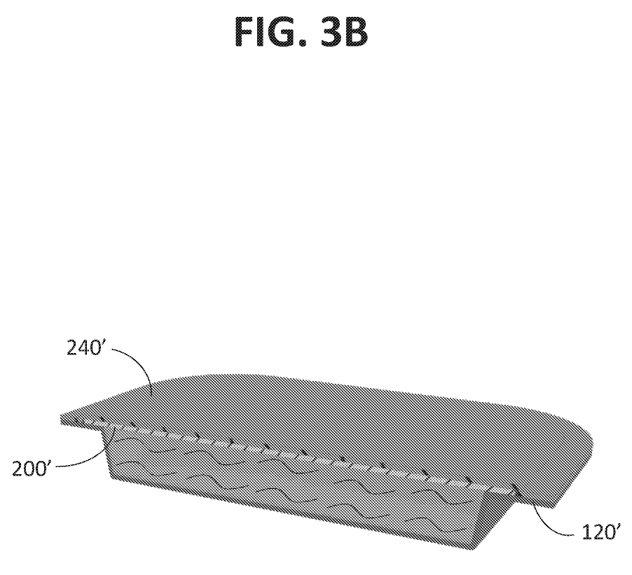

An alternate embodiment of the fragrance delivery device 100′ is depicted in FIG. 3A. In this embodiment, the device includes a flexible impermeable barrier 300′ as a preformed rigid tray, which forms a perimeter seal 120′ with the upper vapor releasing membrane 200′. An edge/lip 301′ of the rigid tray extends beyond the vapor releasing membrane to allow for the attachment of an additional sealing layer 240′. The sealing layer 240′ can comprise for example, a heat sealable foil material disposed over the vapor releasing membrane to prevent ambient vapor loss during storage in warm temperatures. Such sealing layer is removable by a user prior to use. The device of FIG. 3B is similar to the device of FIG. 3A except that the sealing layer 240′ is disposed adjacent the membrane 200′. In this embodiment, the flange of the 120′ extends further than the embodiment of FIG. 3A to allow for the sealing layer to couple thereto. As such, the sealing layer has a greater dimension and surface area than the membrane, as shown.

The substance 400 comprises a volatile fragrance 410 that is disposed in a matrix 420. The volatile fragrance 410 is releasable from the matrix 420 when exposed to the predetermined temperature, such that the matrix retards the release of the volatile fragrance therein. The predetermined temperature ranges from approximately 110° F. to approximately 200° F., which is produced by exposing the fragrance delivery device to the warming unit. In particular, the predetermined temperature ranges between approximately 130° F. to approximately 170° F. With the increase in the predetermined temperature, the substance becomes less viscous and progressively thinner as the matrix provides less resistance to the volatile fragrance. As such, the substance 400, such as a gel substance, has a first viscosity in the delivery condition and a second viscosity in the use-condition. The first viscosity of the gel substance in the delivery condition is greater than the second viscosity of the gel substance in the use-condition. The first viscosity is of such value that locks the volatile fragrance within the matrix and prevents the volatile fragrance from releasing from the matrix. As such, the matrix and releasing membrane collectively and uniformly control an activation of the fragrance delivery device. The matrix alters the rheology of the volatile fragrance by changing the consistency of the volatile fragrance in the delivery condition and extending the volatile fragrance by providing more mass to embed the fragrance therein, as further discussed herein.

In the use-condition, the flexible impermeable barrier 300 acts as barrier and prevents volatile fragrance from releasing there through. As such, the flexible impermeable barrier maintains its structural integrity when exposed up to at least to the range of predetermined temperatures. As such, as the volatile fragrance releases from the matrix as the temperature within the cavity increases, phase change of the volatile fragrance into a vapor state within the cavity forces the volatile fragrance to emit through the only available exits, that being the plurality of micropores of the vapor releasing membrane.

As shown in the fragrance delivery system 111 of FIG. 4A, the device 100 is placed in a warming unit 600, and in particular a dish 610 of the warming unit. In one embodiment, the dish can comprise any suitable material to transfer energy to the fragrance delivery device, such as but not limited to plastic, metal, glass or glazed ceramic. In embodiment of FIG. 4A, the heating device of the warming unit is a light bulb as shown in the cross-sectional view of FIG. 4B. However, other heating devices are contemplated in this application as known in the industry, as further discussed herein. The warming unit emanates energy to the fragrance delivery device at the predetermined temperature. Suitable warming units can be utilized with the device 100 as known in the art, such as but not limited to those devices described in U.S. Publication No. 2005/0016985, U.S. Publication No. 2015/0283280, U.S. Publication No. 2015/0174278, and U.S. Pat. No. 7,067,772, the contents of each of which are incorporated herein by their entireties.

The heat assisted release of the volatile fragrance from the matrix is substantially more significant than that which might occur at elevated ambient temperatures as the substance can collectively transition into a less viscous state from the delivery condition to the use-condition. Accordingly, energy from the warming unit is transferable to the flexible impermeable barrier to warm the substance therein, and alter a viscosity of the substance from the first viscosity to the second viscosity.

In the use-condition, the vapor pressure of the volatile fragrance composition within the substance is greater than the vapor pressure of the matrix material. This “diffusion temperature,” which is the predetermined temperature at which the volatile fragrance enters a vapor state for eventual permeation through the vapor permeable membrane and release from the matrix, can only be reached if there is an external heat source that raises the substance temperature to that needed to induce this phase transition in the use-condition. The operating temperature for typical warming units can range from approximately 110° F. to approximately 200° F. These temperatures are sufficiently high so as to aid in the vapor formation of the most commonly preferred volatile actives.

Once the heat source of the warming unit is shut off, the substance reverts back to the delivery condition state in ambient temperature. The device, once depleted of volatile fragrance, can be disposed of, by simply tipping the warming unit over to transfer the device 100 into a waste receptacle, without ever requiring the user to touch the device. Additionally or alternatively, the fragrance delivery device can be handled directly by the user, such as about the flange 120, as the device is cool enough to touch. In contrast to the disclosed subject matter, currently available wax melts have to first cool in order to be physically chipped out or scooped out of the warming dishes once the fragrances therein are depleted and before a user can introduce another wax melt into the warming unit. Additionally, the present device may be removed from the warming unit at any time in its use cycle. As such, the present device can be removed while the warming unit remains on, and can be changed out in favor of another fragrance type. The initial device can be reintroduced to the warming unit when the user decides to return to the fragrance of the initial device. Thus, the fragrance delivery device is a self-contained unit. Such flexibility does not exist with the currently available wax melt formats.

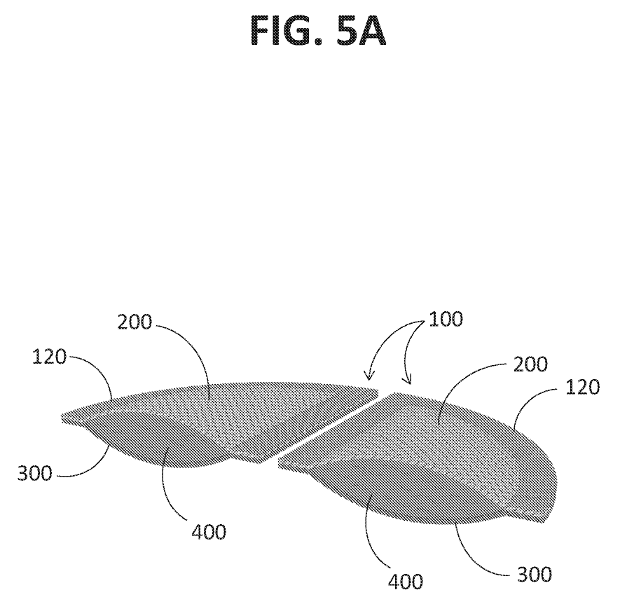

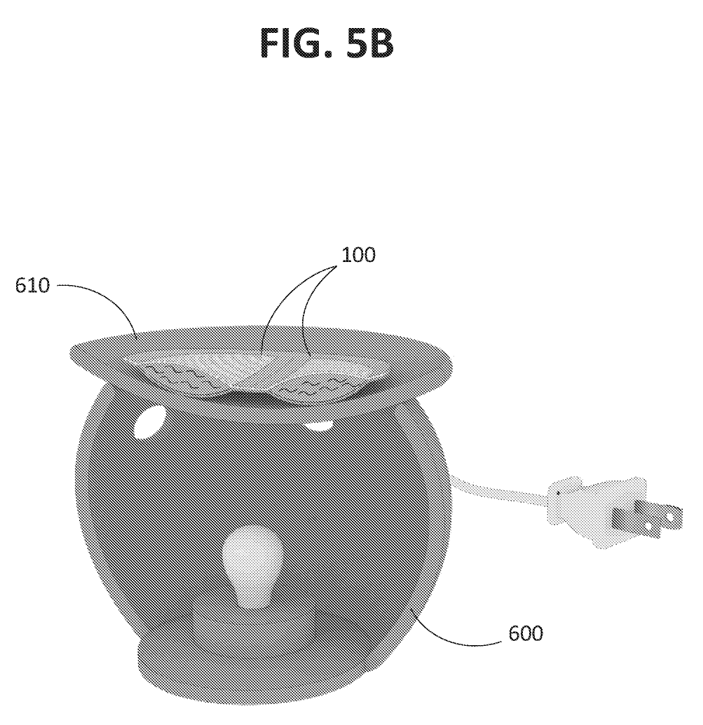

As previously discussed, the fragrance delivery device can comprise any suitable size to be utilized with a warming device. In yet another embodiment as shown in system of FIGS. 5A and 5B, the device may be formed in a reduced size format such that a plurality of fragrance delivery devices, such as two devices 100, interface with a single warming device, such as a dish 610. In this embodiment, the user can create personalized fragrance combinations of their own choosing giving them creative control over the desired fragrance experience, which is not available with currently available wax melts. Further, with utilizing a reduced size delivery device, the intensity of the fragrance can be controlled to meet the needs of a user and to better customize the odor experience of the user, which can be beneficial in utilizing the delivery device within smaller spaces or confines. Such control is unavailable with wax melts. The devices can also be sold together as a unit with complementary pairs of fragrances respectively contained therein, such as a first device having an orange fragrance and the second device containing a vanilla fragrance for a delivery device unit having an orange-vanilla fragrance experience when in the use condition.

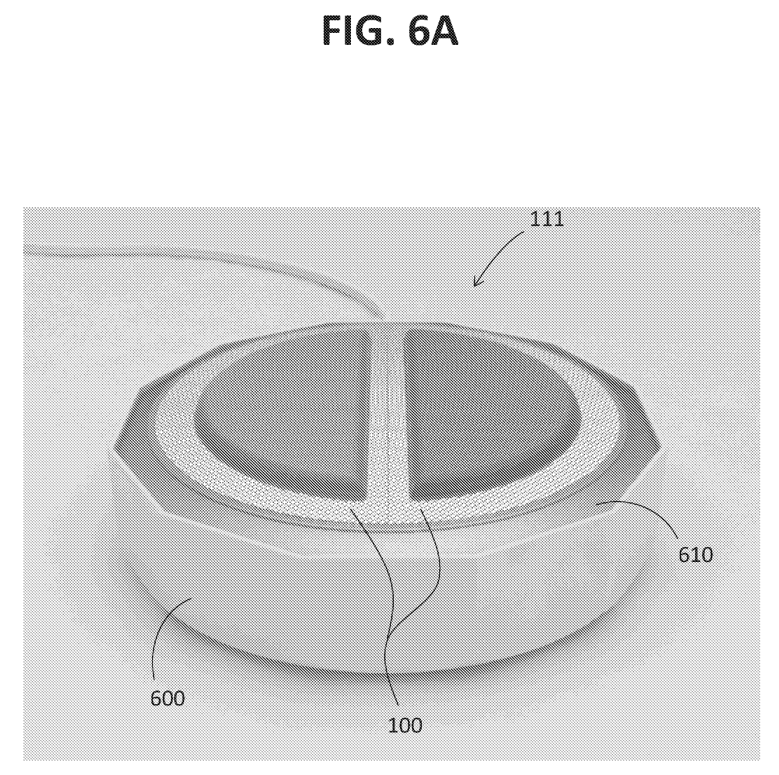

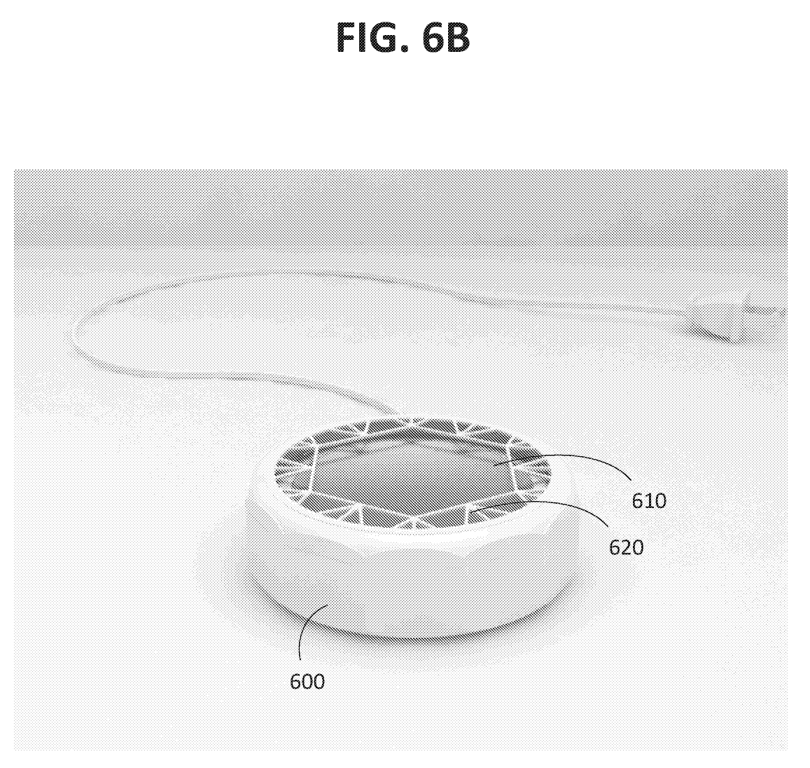

The two devices 100 of FIGS. 5A and 5B can be coupled to each other in the delivery condition and selectively separable upon use. As such, for example and not limitation, the two devices can include a perforation line or the like to couple the devices together or can be integrally manufactured without a separation device. Alternatively, these devices can be manufactured separately in reduced size format, as shown in FIG. 6A.

### Barrier Layer

In the embodiment shown in FIGS. 1A, 1B and 2A-2C, the flexible impermeable barrier is constructed using a flexible, impermeable barrier laminate. In an embodiment of the disclosed subject matter, the barrier laminate comprises a foil layer in a multi-layer construction, which provides an impervious barrier as well as a heat sealable layer of polyethylene or polypropylene. The barrier layer can include any suitable material such as the barrier panel materials as disclosed in U.S. Publication No. 2014/0048614, incorporated herein by reference in its entirety.

The flexible impermeable barrier can comprise any suitable material or materials. In one embodiment, the flexible impermeable barrier comprises at least three layers having an outer layer, a middle layer, and a sealing layer. The outer layer can comprise at least one of polyethylene terephthalate (PET) film, low density polyethylene, medium density polyethylene, high density polyethylene, polypropylene, polyvinyl chloride and blends thereof. The middle layer can comprise at least one of aluminum foil and metalized poly(ethylene terephthalate)-(MET-PET). The sealing layer can comprise at least one of a linear low density polyethylene (LLDPE), low density polyethylene, medium density polyethylene, high density polyethylene, polypropylene, and blends thereof. In another embodiment, the impermeable barrier can include five layers.

In the embodiment shown in FIG. 3A, the barrier layer is formed of a semi-rigid or rigid material, for example but not limitation, polyethylene terephthalate or polyolefin coated aluminum, and is joined to the vapor releasing membrane as known in the art, such as, but not limited to, through the use of a mechanical union or through the means of a sonic or heat sealed weld, forming a fluid tight perimeter seal.

### Vapor Releasing Membrane

The vapor releasing membrane can comprise any suitable material to facilitate at least the two functions noted above (i.e. to inhibit the volatile fragrance from emitting through the plurality of micropores in the delivery condition and to permit the volatile fragrance to be emittable through the plurality of micropores in the use-condition) in addition to being of suitable construction to seal with the flexible impermeable barrier along the perimeter of the device. In certain embodiments, the two layers are sonically or heat sealed. However, other ways to seal the fragrance delivery device are contemplated herein as noted above.

The permeable vapor releasing membrane comprises a microporous material, thereby allowing for the rate of fragrance release into the environment to be controlled via the overall thickness of the material and mean pore diameter, i.e., porosity, of the material. The use of a microporous material enables the presently disclosed fragrance delivery device to offer performance advantages, such as a more linear and predetermined rate of release and a prolonged functional life as compared to currently available wax melts, which result in fully exposed pools of hot molten wax upon being heated. The porosity of the vapor releasing membrane can be adjusted based on the weight and thickness of the microporous materials used.

In one embodiment, the rate controlling vapor permeable membrane is a microporous membranes sold commercially as CELGARD® (Celgard LLC, Charlotte, N.C.). The CELGARD® membrane family is manufactured in a variety of offerings as the porosity and thickness of the membranes offered vary. Particularly suited to the disclosed subject matter is CELGARD® 4560, which is a composite structure comprising CELGARD® 2500, which is laminated to a loosely structured non-woven polypropylene article. CELGARD® 2500 is a hydrophobic polypropylene in which sub-micron pores are formed in the polypropylene film during processing. CELGARD® 2500 has a film thickness of 25 μm while the laminated thickness is approximately 110 μm. The average pore size is approximately 0.064 μm with a porosity of approximately 55% and a Gurley permeability rating of approximately 200 seconds.

FIG. 9A depicts a perspective view of the fragrance delivery device, according to the disclosed subject matter. In this embodiment, and for purposes of illustration, the vapor releasing membrane comprises CELGARD® and the micropores are visible along a surface area thereof. It is noted that the micropores are not drawn to scale and shown throughout in the figures for purposes of illustration.

An additional embodiment of the disclosed subject matter utilizes a non-woven substrate, such as a polypropylene non-woven, coupled with the vapor releasing membrane on the inner side of the cavity in contact with the substance/composition as shown in FIG. 2C. The non-woven can be any suitable material that can be coupled with the vapor releasing membrane and is sealable with the flexible impermeable barrier, such as but not limited to polypropylene, polyolefin, polyester, and blends thereof The non-woven substrate can be heat sealed to the flexible impermeable barrier about a perimeter of the fragrance delivery device. In one embodiment, the substance is anhydrous. As such, the substance is lipophilic and easily compatible with polyolefin non-wovens, inclusive of polypropylene and polyester nonwovens. The sub-micron openings in the CELGARD® vapor permeable membrane allow for a path through the monolithic sheet of the polypropylene membrane such that the active agents in their vapor phase, can permeate the vapor releasing membrane for their eventual release into the air or external environment. By selecting a CELGARD® membrane with a different porosity and/or thickness, the membrane can function as a rate controlling mechanism in exerting influence over the rate of release of the active volatile agents. As such, in addition to fragrance delivery or alternative thereto, the substance can further comprise insecticides and medicants.

In certain embodiments, the vapor releasing membrane comprises a polyolefin based material, which demonstrates good qualities for heat sealing capabilities, thereby allowing for an easily sealed perimeter during manufacture of the device of the disclosed subject matter. Non-limiting examples of microporous polyolefin based materials suitable for use in the present device include, but are not limited to a polyolefin/silica microporous polyethylene films available under the trade name Teslin® (PPG Industries, Pittsburgh, Pa.). The Teslin material is available in sheet stock that can range in thickness from approximately 2 to approximately 18 mils and can have a void volume of approximately 65%.

Additionally, another microporous membrane composition that might be suitable with the present invention includes a spun bonded polyolefin film, of the type commercially sold under the trade name of Tyvek® (E.I. du Pont de Nemours and Company, Wilmington, Del.). Furthermore, the vapor releasing membrane can comprise any suitable materials to meet the previously noted needs. Teslin® membranes, in particular, are compatible with a broad range of print processing and may be utilized in embodiments of the present device intended to have a printed design.

As depicted in FIG. 1A-B and FIG. 9A-B, the vapor releasing membrane can be printed on with text, designs, logos, symbols, warning signs, and the like. As such, if desired, warning messages such as “do not ingest” or “this side up” can be printed directly on the vapor porous membrane to signify a message to a user. The device of FIGS. 1A-B and 9A-9B includes text “Lavender Fields” to signify the scent of the fragrance contained therein. As such, the consumer understands which scent is packaged in the device without having to rely on a color of the substance therein, unlike wax devices. The printing of any colors, text, and designs and the like remain stable when in contact with heat exposure during the use-condition. The functional and aesthetic contributions offered by this print option make the currently disclosed fragrance delivery device more superior than traditional wax melts, which can only be differentiated by color. Although the printing can block the microporous openings in the membrane depending on the material used for printing, a lightly designed pattern or design will not cause any significant reduction in the ability of the fragrance delivery device to release its active agents. Substance

The cavity formed by the sealing of the flexible impermeable barrier and the vapor releasing membrane may be filled with a substance, such as a gel substance, comprising a volatile composition (i.e., fragrance, insecticide, medicament, and the like) dispersed in a matrix material. The volatile composition can be selected from a variety of suitable options including fragrances, aroma therapeutic compositions, medicants, decongestants, insect repellants, insecticides and the like.

With embodiments having a gel substance, the cavity of the present device can accept a wide variety of forms for the gel substance, including semi-solids and high viscosity substances. Non-limiting examples of gel substance options include, but are not limited to metallic soap based gels, elastomeric gels, gels formed using modified clays, e.g., Bentonite gels, or colloidal silica gels. The final consistency of the gel substance composition is highly impacted by the amount of volatile composition dispersed within the matrix. The rheological format of the gel substance should be such that at the operating temperature of the warming unit(s) for which it is intended to be used in the use-condition, the gel substance transitions into a less viscous substance while remaining confined within the cavity of the device.

In other embodiments of the disclosed subject matter, the substance comprises certain waxes, such as but not limited to paraffin waxes, soy waxes, wax blends, wax and oil blends.

Conventional wax melts and wax tarts must be sufficiently solid in order to retain their shape. For this reason, fragrance loadings are kept low, with a range of approximately 6% to approximately 15%, by weight, being common, and with an inability to exceed a maximum of approximately 18% by weight. In contrast, as the fragrance medium of the present device is not limited to a solid, shaped composition, the volatile fragrance loadings can include up to about 40% by weight of the gel substance. As such, the gel substance can comprise approximately 60% to approximately 95% by weight of the gel matrix, and approximately 5% to approximately 40% by weight of the volatile fragrance. In one embodiment in particular, the gel substance can comprise approximately 80% by weight of the gel matrix, and approximately 20% by weight of the volatile fragrance. Since most volatile fragrance compositions tend to be highly plasticizing, when a fragrance load of 10% by weight is added to a solid matrix, the matrix might remain a solid with such 10% by weight loading at ambient temperatures according to the disclosed subject matter. With a fragrance loading of approximately 20% in the same gel matrix, the matrix may have a semi-solid or viscous gel consistency. The reservoir rheology along with the fragrance loading must be balanced in such a way that the fluid nature of the gel substance composition at elevated temperatures in the use condition, or even at ambient temperatures in the delivery condition, does not permeate the micropores of the vapor releasing membrane and result in a fluid accumulation on the outside of the fragrance delivery device.

As the device is heated in the warming unit, the gel substance transitions into a lower viscosity substance. However, such lower viscosity substance remains sealed within the cavity and in contact with the vapor releasing membrane such that the volatile composition is able to release from the gel matrix and emit through the micropores of the membrane without the lower viscosity substance exiting there through.

In one embodiment, the gel matrix comprises at least one of napthenic oil, isoparaffinic solvent, a phenolic antioxidant, elastomeric polymer, and mixtures thereof. For example, the gel matrix can include the following composition and range of percentages by weight:

| Ingredient: | Range % |
|---|---|
| Calsol 5550 Napthenic Oil | appx 20%-appx 60% |
| Isopar V Isoparaffinic Solvent | appx 5%-appx 50% |
| Isopar M Isoparaffinic Solvent | appx 5%-appx 30% |
| Irganox 1010 antioxidant | appx 0.1%-appx 0.5% |
| Kraton G1652 Elastomeric Polymer | appx 10%-appx 45% |

In one embodiment, the gel matrix has a viscosity ranging approximately 3600 cps to approximately 5000 cps at a temperature of approximately 200° F. and specific gravity of approximately 0.92. As the temperature elevates, the viscosity of such gel matrix can decrease to approximately 2100 cps to approximately 3000 cps at a temperature of approximately 210° F. and specific gravity of approximately 0.92. As the temperature further elevates, the viscosity of said gel matrix further decreases to a range of approximately 1350 cps to approximately 1900 cps at a temperature of approximately 220° F. and specific gravity of approximately 0.92. As such, the characteristics of the gel matrix demonstrate the effect that the viscosity thereof decreases as the temperature applied thereto increases.

Once a fragrance is added to the gel, as previously described, there is a plasticizing effect by the components of the fragrance upon the gel. For purposes of example, and not limitation, a gel composition whose matrix includes the viscosity figures are disclosed above at the varying temperatures between 200° F. to 220° F. and has a volatile fragrance that contains 20% by weight of a “clean linen” type fragrance, the resulting viscosity of the gel composition includes a viscosity of approximately 71,000 cps at 75° F., a viscosity of 773 cps at approximately 130° F., a viscosity of approximately 680 cps at approximately 160° F., and a viscosity of approximately 637 cps at approximately 190° F. In another embodiment where the gel substance has been loaded with a volatile fragrance of approximately 20% by weight, the first viscosity of the gel substance ranges from approximately 40,000 cps to approximately 75,000 cps, and the second viscosity of the gel substance ranges from approximately 500 cps to approximately 1000 cps.

### Packaging

The fragrance delivery device can be delivered in any suitable packaging to contain the device therein. As such, for example, when the devices are inadvertently stored in warehouses that reach temperatures greater than 110° F., any release of the volatile fragrance therefrom will be contained within the packaging. In one embodiment, the fragrance delivery device is packaged in a polyester film pouch, but other packaging devices are further contemplated herein.

### Use of the Device

In accordance with another aspect of the disclosed subject matter, a method of using a fragrance delivery system is further disclosed. As previously disclosed, the fragrance delivery device can be provided as further discussed herein, along with a warming unit. The fragrance delivery device can interface with the warming unit, such as being deposited into the dish of a warming unit. The fragrance delivery device can be exposed to the warming unit to emanate energy to the flexible impermeable barrier of the fragrance delivery device at a predetermined temperature. The volatile fragrance is releasable from the gel matrix when exposed to the predetermined temperature and is emittable through the plurality of micropores of the vapor releasing membrane in the use-condition.

The fragrance delivery device can be subsequently removed from the warming unit, such as upon the depletion of the volatile fragrance from the substance or as desired. A second fragrance delivery device can subsequently interface with the warming unit, such as deposited into the dish of the warming unit, as shown in FIG. 5B. With embodiments as depicted in FIGS. 5A and 5B, the second fragrance delivery device can be deposited into the dish of the warming unit and adjacent a first fragrance delivery device to create a personalized fragrance experience. In one embodiment, at least one of the first or second fragrance delivery devices can comprise a medicant, decongestant, insect repellant, and/or insecticide, whereas the other fragrance delivery device can include a fragrance or an aroma therapeutic composition.

FIG. 6A depicts another embodiment of a fragrance delivery system having a first and second fragrance delivery device that interface with a warming unit. As shown, the outer housing of the warming unit can have any suitable configuration that does not need to correspond with the shape of the fragrance delivery device(s). FIG. 6B depicts another warming unit according to the disclosed subject matter. In this embodiment, a grille 620 is disposed over the warming dish 610 that can serve as a decorative element in addition to preventing accidental contact with the warming dish. The grille is disposed above and distanced from the warming dish, as shown.

In accordance with the disclosed subject matter, the warming units or “warmers” commercially available for use with wax melts may be used with the presently described device. Examples of such warming units include, but are not limited to, those described in U.S. Publication No. 2014/0048614, incorporated herein by reference in its entirety. Such warming units may be heated by any number of sources, including for example, a resistance heater, a tea light candle or other candles, a light bulb, and devices containing exothermic chemical reactions to induce an elevated temperature, amongst other known units as understood in the industry. With devices containing exothermic chemical reactions, such warming unit can be independent of electrical outlets and plugs that can be especially useful when using a delivery device according to the disclosed subject matter contains an insect repellent for use outdoors. However, any heat source that is sufficient to convert the volatile composition to release through the micropores is contemplated herein.

In accordance with another aspect of the disclosed subject matter, a method of manufacturing a fragrance delivery device is provided, comprising: providing vapor releasing membrane including a plurality of micropores, wherein each respective micropore has a consistent diameter dimension in both a delivery condition and in a use-condition of the fragrance delivery device; coupling a flexible impermeable barrier to the vapor releasing membrane, wherein the vapor releasing membrane and impermeable barrier define a cavity there between having a predetermined volume; and disposing a substance in the cavity up to the predetermined volume, the substance comprising a volatile fragrance disposed in a matrix; and sealing the vapor releasing membrane with the flexible impermeable barrier at a perimeter thereof, wherein the substance has a first viscosity in the delivery condition and a second viscosity in the use-condition, the volatile fragrance being releasable from the matrix when exposed to the predetermined temperature and emittable through the plurality of micropores in the use-condition.

The substance can be manufactured in any suitable manner to facilitate the volatile fragrance being contained within the matrix. In one embodiment, the matrix is a gel matrix made separately and then heated to a liquefied form to which the volatile fragrance is added at a desired weight percentage, as further discussed herein. As there is an inherent tendency for the volatile fragrance to bleed or sweat through the pores without the use of a matrix, the substance having the volatile fragrance with the matrix permits the manufacture of a larger fragrance delivery device with a greater surface area, thereby increasing the surface area of the emanating surface, resulting in a greater fragrance diffusion.

### EXAMPLES

The presently disclosed subject matter will be better understood by reference to the following Examples, which are provided as exemplary of the disclosure, and not by way of limitation.

### Example 1

### Fragrance Intensity Study

Example 1 provides the results from a study that tested the devices of the presently disclosed subject matter. In particular, the study was to compare the intensity of fragrance release over time of the present device according to an embodiment of the disclosed subject matter, as compared to a standard wax melt.

Results of the study are shown in Table 1.

TABLE 1

| | Intensity (1 min to 10 max) | | | | | |
|---|---|---|---|---|---|---|
| | 4 | 8 | 12 | 16 | 20 | |
| Fragrance | Hours | Hours | Hours | Hours | Hours | 25 Hours |
| Apple Cinnamon (known | 10 | 9 | 9 | 9 | 5 | 3 |
| Commercial Wax Melt) | | | | | | |
| Apple Cinnamon (Delivery | 10 | 10 | 10 | 10 | 7.5 | 6 |
| Device with Celgard | | | | | | |
| Membrane) | | | | | | |
| Cherry - Fruity Bouquet | 9 | 8 | 6 | 5 | 5 | 5 |
| (known Commercial Wax | | | | | | |
| Melt) | | | | | | |
| Cherry - Fruit Bouquet | 9 | 9 | 8 | 8 | 7 | 7 |
| (Delivery Device with Celgard | | | | | | |
| Membrane) | | | | | | |
| Tropical - Fruity Bouquet | 9 | 9 | 9 | 7 | 5 | 5 |
| (known Commercial Wax | | | | | | |
| Melt) | | | | | | |
| Tropical - Fruity Bouquet | 9 | 9 | 9 | 8 | 7 | 7 |
| (Delivery Device with Celgard | | | | | | |
| Membrane) | | | | | | |
| Peach Bouquet (known | 10 | 9 | 8 | 6 | 5 | 5 |
| Commercial Wax Melt) | | | | | | |
| Peach Bouquet (Delivery | 10 | 10 | 10 | 9 | 9 | 8 |
| Device with Celgard | | | | | | |
| Membrane) | | | | | | |

Devices of the disclosed subject matter were fabricated with a Celgard 4560 membrane as depicted in FIG. 1A that had a surface area of 5 square inches, filled with 8.0 grams of an elastomeric gel substance composition as loaded with 20% by weight of each of the fragrances shown in Table 1. Standard, commercially available wax melts available in approximately the same fragrance family as those fragrances utilized in the fabricated devices were obtained and tested against the disclosed devices. Each of the fragrance delivery devices according to the disclosed subject matter and commercially available wax melts were placed in warming units and allowed to age for a period of 20 minutes prior to being placed in odor evaluation rooms. After which, the devices were placed in odor evaluation rooms for the times designated in Table 1. Odor assessment tests were run on the samples in 800 cubic feet odor evaluation rooms. Each of the samples was allowed to equilibrate in the odor room for 20 minutes prior to being evaluated. The samples were subsequently evaluated by a panel of expert evaluators on an intensity scale ranging from 10 (highest intensity, i.e. most fragrant) to 1 (lowest intensity, i.e. not as fragrant).

The results demonstrate that devices of the presently disclosed subject matter retain more consistent fragrance intensity over the course of the testing than the standard commercially available wax melts.

### Example 2

### Fragrance Intensity Study

Example 2 provides the results from another study that tested the devices of the presently disclosed subject matter in comparison with commercially available wax melts. In particular, the study was to compare the intensity of fragrance release over time of the present device from delivery condition to use condition according to an embodiment of the disclosed subject matter, as compared to a standard wax melt as provided in its delivery condition to use condition.

Results of the study are shown in Table 2.

TABLE 2

| | Odor intensity Score (Scale 1-10) | | |
|---|---|---|---|
| Elapsed | | | Microporous |
| Time | Wax Melt | Wax Melt | Membrane Unit |
| (minutes) | (7% Loading) | (18% Loading) | (20% Loading) |
| 0 | 0 | 0 | 0 |
| 5 | 0 | 1 | 4 |
| 10 | 1 | 3 | 7 |
| 15 | 5 | 6 | 8 |
| 20 | 7 | 8 (Wax | 9 |
| | | Completely Melted) | |
| 25 | 7 (Wax | 8 | 9 |
| | Completely Melted) | | |

Devices of the disclosed subject matter as depicted in FIG. 1A were tested that had a surface area of 5 square inches, filled with 8 grams of a gel composition as loaded with 20% by weight of a volatile fragrances shown in Table 2. The device was placed in a warming unit as shown in FIG. 4A. Standard, commercially available wax melts available in approximately the same fragrance family as the fragrance of the device were utilized in this intensity study and were placed in a warming device whose tray allowed for the molten wax to collect therein, and the dish approximately had a dimension of 6 square inches. The devices according to the disclosed subject matter were tested against such standard wax melts. A first wax melt having a loading of approximately 7% by weight of fragrance was tested (the balance of which being 93% by weight of wax), a second wax melt having a loading of approximately 18% by weight was tested (the balance of which being 82% by weight of wax), along with a device according to the disclosed subject matter as shown in FIG. 1A with a loading of 20% volatile fragrance was tested (the balance of which being 80% gel matrix) Each of the fragrance delivery devices according to the disclosed subject matter and commercially available wax melts were placed in warming units in their respective delivery condition and placed in rooms to determine the intensity of the fragrance with respect to time designated in Table 2 without an equilibrium time that was previously provided with respect to Example 1.

Odor assessment tests were run on the samples in an 800 cubic feet odor evaluation rooms and evaluated by a panel of expert evaluators on an intensity scale ranging from 10 (highest intensity, i.e. most fragrant) to 1 (lowest intensity, i.e. not as fragrant). Each of the samples was placed on a warming unit and were immediately evaluated at the designated times noted in Table 2.

The results of Table 2 demonstrate that devices of the presently disclosed subject matter emitted volatile fragrance and had more notable fragrance intensity at a much faster, quicker rate than the standard commercially available wax melts.

### Example 3

### Weight Loss

The weight loss of the fragrance delivery device can be determined in the delivery condition and after the use-condition when the volatile fragrance has been depleted. The volatile fragrance is fugitive along with a select amount of the gel matrix. In one example, the gel substance comprises an amount of 8 g, wherein approximately 1.6 g. is volatile fragrance and 6.4 g. is gel matrix. After the use-condition when the volatile fragrance has been depleted, the amount of gel substances left in the cavity is approximately 5.2 g. However, over a 25 hour functional life cycle of the fragrance delivery device, depending on the type of fragrance, a loss of the gel substance can be about 2.5 g, or range from about 1.5 g to about 3.0 g loss, or up to approximately 31% of the gel substance weight.

Although the presently disclosed subject matter and its advantages have been described in detail, it should be understood that various changes, substitutions and alterations can be made herein without departing from the spirit and scope of the application as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, means, methods, or steps.

In addition to the various embodiments depicted and claimed, the disclosed subject matter is also directed to other embodiments having any other possible combination of the features disclosed and claimed herein. As such, the particular features presented herein can be combined with each other in other manners within the scope of the disclosed subject matter such that the disclosed subject matter includes any suitable combination of the features disclosed herein. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the device, method and system of the disclosed subject matter without departing from the spirit or scope of the disclosed subject matter. Thus, it is intended that the disclosed subject matter include modifications and variations that are within the scope of the appended claims and their equivalents.

For any patents, patent applications, publications, product descriptions, and protocols are cited throughout this application, the disclosures of all of which are incorporated herein by reference in their entireties for all purposes.

1. A fragrance delivery device, comprising:
a vapor releasing membrane including a plurality of micropores, wherein each respective micropore has a consistent diameter dimension in both a delivery condition and in a use-condition of the fragrance delivery device;
a flexible impermeable barrier coupled to the vapor releasing membrane along a perimeter thereof, wherein the vapor releasing membrane and impermeable barrier define a cavity there between having a predetermined volume; and
a substance disposed in the cavity up to the predetermined volume, the substance comprising a volatile fragrance disposed in a matrix, wherein the substance has a first viscosity in the delivery condition and a second viscosity in the use-condition, the volatile fragrance being releasable from the matrix when exposed to a predetermined temperature and emittable through the plurality of micropores in the use-condition.

2. The fragrance delivery device of claim 1, wherein the vapor releasing membrane inhibits the volatile fragrance from emitting through the plurality of micropores in the delivery condition.

3. The fragrance delivery device of claim 1, wherein the diameter dimension of each respective micropore comprises approximately 0.06 μm.

4. The fragrance delivery device of claim 1, wherein the substance comprises a gel substance, and the matrix comprises a gel matrix.

5. The fragrance delivery device of claim 4, wherein the gel substance comprises approximately 60% to approximately 95% by weight of the gel matrix and approximately 5% to approximately 40% by weight of the volatile fragrance.

6. The fragrance delivery device of claim 5, wherein the gel substance comprises approximately 80% by weight of the gel matrix and approximately 20% by weight of the volatile fragrance, and wherein the first viscosity of the gel substance ranges from approximately 40,000 cps to approximately 75,000 cps, and the second viscosity of the gel substance ranges from approximately 500 cps to approximately 1000 cps.

7. The fragrance delivery device of claim 4, wherein the first viscosity is greater than the second viscosity.

8. The fragrance delivery device of claim 4, wherein the gel matrix has a viscosity ranging approximately 3600 cps to approximately 5000 cps at a temperature of approximately 200° F. and specific gravity of approximately 0.92 and has a viscosity ranging approximately 2100 cps to approximately 3000 cps at a temperature of approximately 210° F. and specific gravity of approximately 0.92.

9. The fragrance delivery device of claim 4, wherein the gel matrix comprises at least one of napthenic oil, isoparaffinic solvent, a phenolic antioxidant, elastomeric polymer, and mixtures thereof.

10. The fragrance delivery device of claim 1, wherein the substance comprises at least one of a paraffin wax, soy wax, wax blend, and a wax and oil blend.

11. The fragrance delivery device of claim 1, wherein the flexible impermeable barrier maintains structural integrity when exposed up to at least to the predetermined temperature.

12. The fragrance delivery device of claim 1, wherein the flexible impermeable barrier comprises at least three layers having an outer layer, a middle layer, and a sealing layer.

13. The fragrance delivery device of claim 12, wherein the outer layer comprises at least one of polyethylene terephthalate (PET) film, low density polyethylene, medium density polyethylene, high density polyethylene, and polypropylene;
the middle layer comprises at least one of aluminum foil and metalized polyethylene terephthalate; and
the sealing layer comprises at least one of a linear low density polyethylene (LLDPE), low density polyethylene, medium density polyethylene, high density polyethylene, polypropylene, and blends thereof.

14. The fragrance delivery device of claim 1,wherein energy is transferable to the flexible impermeable barrier to warm the gel substance therein and alter a viscosity of the gel substance from the first viscosity to the second viscosity.

15. The fragrance delivery device of claim 1, wherein the predetermined temperature ranges from approximately 110° F. to approximately 200° F.

16. The fragrance delivery device of claim 1, wherein the delivery condition is at a temperature below approximately 110° F.

17. The fragrance delivery device of claim 1, wherein the fragrance delivery device is a self-contained unit.

18. The fragrance delivery device of claim 1, wherein the substance comprises the predetermined volume in the delivery condition.

19. The fragrance delivery device of claim 1, wherein the volatile fragrance includes at least one of a medicant, decongestant, insect repellant, insecticide, fragrance, and an aroma therapeutic composition.

20. A fragrance delivery system, comprising:
a fragrance delivery device having
a vapor releasing membrane including a plurality of micropores, wherein each respective micropore has a consistent diameter dimension in both a delivery condition and in a use-condition of the fragrance delivery device,
a flexible impermeable barrier coupled to the vapor releasing membrane along a perimeter thereof, wherein the vapor releasing membrane and impermeable barrier define a cavity there between having a predetermined volume, and
a substance disposed in the cavity up to the predetermined volume, the substance comprising a volatile fragrance disposed in a matrix, wherein the substance has a first viscosity in the delivery condition and a second viscosity in the use-condition; and
a warming unit to emanate energy to the fragrance delivery device at a predetermined temperature, wherein the volatile fragrance is releasable from the matrix when exposed to the predetermined temperature and emittable through the plurality of micropores in the use-condition.

21. The fragrance delivery device of claim 20, wherein the substance comprises a gel substance and the matrix comprises a gel matrix, wherein the first viscosity of the gel substance is greater than the second viscosity of the gel substance.

22. A method of manufacturing a fragrance delivery device, comprising:
providing vapor releasing membrane including a plurality of micropores, wherein each respective micropore has a consistent diameter dimension in both a delivery condition and in a use-condition of the fragrance delivery device;
coupling a flexible impermeable barrier to the vapor releasing membrane, wherein the vapor releasing membrane and impermeable barrier define a cavity there between having a predetermined volume; and
disposing a substance in the cavity up to the predetermined volume, the substance comprising a volatile fragrance disposed in a matrix; and
sealing the vapor releasing membrane with the flexible impermeable barrier at a perimeter thereof, wherein the substance has a first viscosity in the delivery condition and a second viscosity in the use-condition, the volatile fragrance being releasable from the matrix when exposed to the predetermined temperature and emittable through the plurality of micropores in the use-condition.

23. A method of using a fragrance delivery system, comprising:
providing a fragrance delivery device having
a vapor releasing membrane including a plurality of micropores, wherein each respective micropore has a consistent diameter dimension in both a delivery condition and in a use-condition of the fragrance delivery device,
a flexible impermeable barrier coupled to the vapor releasing membrane along a perimeter thereof, wherein the vapor releasing membrane and impermeable barrier define a cavity there between having a predetermined volume, and
a substance disposed in the cavity up to the predetermined volume, the substance comprising a volatile fragrance disposed in a matrix, wherein the substance has a first viscosity in the delivery condition and a second viscosity in the use-condition;
providing a warming unit having a dish;
depositing the fragrance delivery device into the dish of a warming unit; and
exposing the fragrance delivery device to the warming unit to emanate energy to the fragrance delivery device at a predetermined temperature, wherein the volatile fragrance is releasable from the matrix when exposed to the predetermined temperature and emittable through the plurality of micropores in the use-condition.

24. The method of using the fragrance delivery system of claim 23,
comprising removing the fragrance delivery device from the warming unit; and
depositing a second fragrance delivery device into the dish of the warming unit.

25. The method of using the fragrance delivery system of claim 23,
comprising depositing a second fragrance delivery device into the dish of the warming unit and adjacent the fragrance delivery device.
